(19) United States
(12) Patent Application Publication
(10) Pub. No.: US 2019/0112640 A1
(43) Pub. Date: Apr. 18, 2019

(54) METHOD FOR MAPPING SPINAL MUSCULAR ATROPHY (“SMA”) LOCUS AND OTHER COMPLEX GENOMIC REGIONS USING MOLECULAR COMBING

(71) Applicant: GENOMIC VISION, Bagneux (FR)

(72) Inventors: Marjorie PIERRET, Gif Sur Yvette (FR); Sara BERTHOUMIEUX, Paris (FR); Sebastien BARRADEAU, Paris (FR); Aaron BENSIMON, Antony (CH)

(73) Assignee: GENOMIC VISION, Bagneux (FR)

(21) Appl. No.: 15/783,714

(22) Filed: Oct. 13, 2017

(51) Int. Cl. C12Q 1/68; G06F 19/18

(52) U.S. Cl. CPC C12Q 1/6816; C12Q 1/6883; C12Q 2600/158; G06F 19/18; C12Q 1/6806

(57) ABSTRACT

A molecular-combing, Genetic-Morse Code based method enabling the detection and high-resolution characterization of complex regions of genomic DNA, such as the SMA locus, with molecular combing. A method for the identification of biomarkers associated to the cis-duplication of SMN1 gene or segments of other complex parts of the genome. Biomarkers identified by this method which are composed of a sets of different colored probes, such as those disclosed for the SMA region.

### STATEMENT REGARDING PRIOR DISCLOSURES BY THE INVENTOR(S)

Aspects of this technology are described by Pierret, et al., ASHG PgmNr 850/W: Molecular combing reveals structural variations in the Spinal Muscular Atrophy locus in African-American population, Abstract (Oct. 18-22, 2016).

### BACKGROUND

### Field of the Invention

The present invention concerns a process that enables the detection and high-resolution characterization of complex regions of genomic DNA, such as the SMA locus, with molecular combing. Moreover, the invention concerns a method for the identification of biomarkers associated to the cis-duplication of SMN1 gene. It concerns also the biomarkers identified by this method which are composed of a sets of different colored probes.

### Description of Related Art

The “background” description provided herein is for the purpose of generally presenting the context of the disclosure. Work of the presently named inventor(s), to the extent it is described in this background section, as well as aspects of the description which may not otherwise qualify as prior art at the time of filing, are neither expressly or impliedly admitted as prior art against the present invention.

Spinal Muscular Atrophy (SMA) is an autosomal recessive disease characterized by degeneration of the anterior motor neurons, leading to progressive muscle weakness and paralysis. SMA is a leading inherited cause of infant death with a reported incidence of in 6000-10000 live births.

SMA is caused by mutations in the survival motor neuron 1 (SMN1) gene. The SMN1 gene is located in a complex region of 5q13 containing SMN2, a homologous pseudogene of SMN1. SMN1 and SMN2 differ by five nucleotides, one of which is in the coding region, in exon 7. This sequence change affects splicing resulting in reduced expression of full-length functional protein from the SMN2 gene.

The homozygous absence of SMN1, due to deletion or gene conversion (of SMN 1 to SMN2) is responsible for 95% of the SMA cases. SMA carriers typically have 1 normal copy and 1 mutated copy of SMN1, and do not exhibit symptoms. The current diagnosis of SMA and the carrier screening is carried out by dosage analysis of SMN genes and determination of a copy number of SMN1.

By molecular analysis, the SMN locus has been mapped to chromosome 5q11.2-q13.3. The region containing this locus has duplications of a large segment of around 500 kilobases (kb) containing several different genes which are present in telomeric (t) and a centromeric (c) copies as shown in FIG. 1. These genes include SMN1 (or SMNt) and SMN2 (or SMNc), neuronal apoptosis inhibitory protein gene (NAIP and its pesudogene ϕNAIP), Small EDRK-Rich Factor 1A (SERF1A) and its paralog SERF1B, and GTF2H2 (general transcription factor IIH). The SMN locus in 5q region is particularly unstable and prone to large scale deletions. FIG. 1 depicts the organization of the SMA locus including the centromeric and telomeric repeated elements.

Due to its high complexity and its large size, and limitations on conventional MLPA or sequencing methodologies, the genomic organization of the SMN locus is not well-characterized and existing sequence information may contain errors. To better characterize the SMN locus and other complex parts of the genome with similar complexity [Bailey, 2002], the inventors applied Molecular Combing technology to map the SMN locus-down to the kb-scale.

The majority of mutations causing all SMA subtypes involve SMN1 copy-number loss. Consequently, carrier screening must be performed by dosage-sensitive methods that can distinguish SMN1 from SMN2, including quantitative PCR [Feldkötter, 2002], multiplex ligation-dependent probe amplification (MLPA) [Huang, 2007], and/or TaqMan quantitative technology [Anhuf, 2003].

Nguyen, U.S. 2006/0088842 A1 describes RT-based cloning of human SMN and construction of expression plasmids. McCabe, et al., U.S. 2015/0258170 A1 describes diagnosis and treatment of SMA and SMN deficiency by detecting particular proteins. However, none of these established methods can determine the number of SMN1 copies present on an individual chromosome. Individuals with two SMN1 copies on one chromosome (duplication allele) and no copies on the other (deletion allele) are silent (2+0) carriers. In contrast, most individuals with two intact SMN1 copies (one on each chromosome) or (1+1) are not carriers.

As a consequence, SMA carrier detection by current techniques directly or indirectly measuring SMN1 copy number generates false-negative results: two SMN1 copies will be detected for both a (2+0) individual who is a carrier and a (1+1) individual who generally is not.

The frequency of silent (2+0) carriers varies and is directly proportional to the product of the deletion and duplication allele frequencies in a given population. The highest false negative rate has been, observed in African-American population [Hendrickson, 2009, Sugarman, 2012].

The ability to identify silent (2+0) carriers will significantly improve carrier detection. Efforts are being directed to identify ethnic-specific SMN1 founder deletion and/or duplication alleles by detecting a genotype unique to either the deletion or duplication alleles present in silent (2+0) carriers in different populations. Such research has been published for example on Ashkenazi population where founder discovery was performed using microsatellite analysis, see [Luo 2013].

As shown herein, molecular combing associated with direct haplotype phasing of the SMA genetic region for individuals enables the identification of potential biomarkers. Using molecular combing, the inventors show herein the biomarkers of cw-duplication of SMN1 gene obtained on African-American population.

### BRIEF SUMMARY OF THE INVENTION

The design of a specific Genetic Morse Code and use of hybridization of labelling probes associated with molecular combing resulted in the visualization of individual DNA molecules and precise physical mapping of the SMA locus. This was not possible with conventional methodologies. The alignment of the fluorescent array signals to the theoretical pattern of colored probes deduced from the human genome reference sequence GRCh38/hg38 assembly revealed several differences or discrepancies with molecular combing/GMC data obtained from the SMA locus in an African-American population.

First, the two SMN genes were found to be in a tail-to-tail orientation and not in a head to-tail orientation as annotated, with an inversion of the centromeric region comprising SMN, NAIP and SERF genes. Moreover, a color pattern from the theoretical GMC was not observed in African-American individuals indicating the absence of the corresponding sequence. The inventors also identified a repeat sequence consisting of red and blue probes with a variable number of repealed units located at the telomeric and/or centromeric regions indicating the presence of an unknown copy number variation sequence. This CNV was found in all individuals analyzed with a number of repeated units variable from 2 to 15 repeats. The classification of those CNV and the color-coded pattern created with the GMC allow the inventors to characterize precisely the SMA locus and reconstitute the alleles. The allelic reconstitution realized for 48 samples suggested a different organization of the SMA locus depending on the number of SMN genes. As these results show, Molecular Combing is a powerful technology that permits precise and accurate mapping of the SMA locus in an African-American population. This corrected, updated map for this population provides information that will be helpful in the development of a relevant SMA screening test for the African-American population. Moreover, these results clearly demonstrate the advantages of the Molecular Combing compared to conventional technologies like sequencing or MLPA that were not able to precisely map and reconstruct haplotypes of the SMA locus because of the complexity of that genomic region.

A molecular combing approach can be used as a general tool for identification and characterization of complex locus and can bring new information that will be helpful for the understanding of genomic organization and discovery of biomarker for diagnostic development. More precisely, this approach enables the identification of biomarkers for the presence of founder genetic rearrangements in specific populations. The inventors show how molecular combing can be used for identification of biomarkers for the presence of cis-duplication in SMN1 gene in specific ethnic populations. This question is of particular interest due to the important false detection rate in current SMA carrier screening tests as described in the Description of Related Art Section.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Organization of the Spinal Muscular Atrophy (“SMA”) locus with the centromeric and telomeric repeated elements.

FIG. 2. Genetic Morse Code (“GMC”) with relative positions of DNA probes according to GRCh38/hg38 human genome assembly (http://_genome.ucsc.edu/). The relative positions of genes localized on the SMA locus are indicated below the GMC. SMN1 and SMN2 genes are covered by a unique magenta probe. Two BACs are used as flanking probes to orientate the SMA locus: one Survival of Motor Neuron (“SMN”) gene associated with the centromeric red probe (SMN_C), and one SMN with the telomeric blue probe. Top: “Reference” Position of the reference DNA fragments designed on the SMA locus Bottom: “Theoretical” Color-coded pattern form by hybridization of the probes along the SMA locus. (GRCh38/hg38; chr5: 69071065-71594127).

FIG. 3. Alignment of SMA fluorescent arrays with the theoretical GMC and SMA locus reconstitution.

FIG. 4. Distribution of copy number variations (“CNV”).

FIG. 5. Mapping of the 2 SMN alleles from African-American individual with 2 SMN1 and 2 SMN2 copies.

FIG. 6. Mapping of the 2 SMN alleles from African-American individual with 2 copies of SMN1 and no copy of SMN2 genes.

FIG. 7. Scheme of data analysis method for detection of biomarker for SMN1 cis-duplication using molecular combing.

FIG. 8. Presentation of potential biomarkers identified for presence of cis-duplication of SMN1 gene.

FIG. 9. Examples of allelic reconstitution for individuals from Test group.

FIG. 10. Examples of allelic reconstitution for individuals from Control group.

FIGS. 11A-11H graphically depicts the colors of the SMA GMC v3 fragments and describes probes

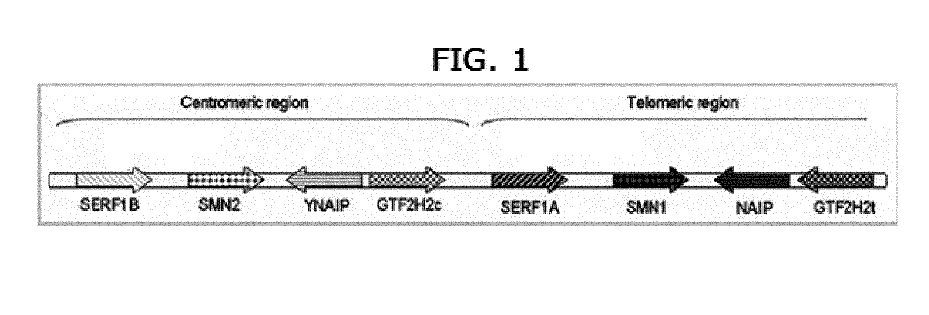

### DETAILED DESCRIPTION OF THE INVENTION

Definitions

Genomic Morse Code Genomic Morse Code or GMC is a tool and method for comprehensive analysis of a physical mapping of one or more target regions on a nucleic acid, such as a target region of a stretched nucleic acid, such as a DNA molecule stretched using molecular combing. GMC probes generally comprise a combination of fluorescent probes of different colors and sizes, designed to recognize a selected region of interest. As a result, the DNA sequence to be analyzed is labelled with the combination of “dashes and dots”, creating a “Morse Code” specific to a target gene and its flanking regions.

Genomic Morse Code provides a comprehensive analysis and physical mapping of target regions on stretched DNA. Combed DNA is hybridized with a combination of fluorescent probes of different colors and sizes, designed to;recognize a selected region of interest. As a result, the DNA sequence to be analyzed is labelled with the combination of “dashes and dots”, creating a “Morse Code” specific to a target gene and its flanking regions. The strategy underlying GMC is to use the, spatial distribution of the probes to provide additional information than simply measuring just the probes. The recognition of different motifs in the Morse Code is not only based on probe size and color, but also on their order and the distances between them. The identical stretching of the DNA allows for accurate and reproducible measurements of the length of the probes as well as the gaps separating them. Any change in the observed pattern compared to the Morse Code of a reference indicates the presence of a rearrangement in the target locus. Amplifications, deletions, repeats, inversions and translocations can be identified and analyzed depending on the chosen Morse Code design with no bias due to sequence content. The GMC method allows the detection of balanced rearrangements often missed by other methods and also provides information about the location and the exact number of copies found. The invention provides GMC probes specifically designed to cover the SMA region.

Known methods for designing and making GMC probes and molecular combing procedures are described by US 2016/0047006, US 2016/0040249, US 2016/0040220, US 2015/0197816, US 2014/0220160, US 2013/0130246, and US 2012/0076871, US 2011/0287423, US 2010/0041036 (now US Pat. No. 8,586,723) and US 2008/0064144 (now US Pat. No. 7,985,542) each of which is incorporated by reference.

The term Genomic Morse Code may be used in conjunction with the set of probes that when bound to a target locus or loci produce a particular pattern of colors or particular detectable labelling pattern or, alternatively, to identify the color or detectable label pattern exhibited by a target nucleic acid contacted with these probes. This term also encompasses the definitions of Genetic Morse Codes used in U.S. Pat. No. 8,586,723 (issued 2013) and U.S. Pat. No. 7,985,542 (issued 2011),

Molecular Combing. Molecular combing techniques are known in the art, including those incorporated by, reference to Bensimon, et al., U.S. Pat. No. 6,248,537 B1 and to Bensimon, et al., EP 1 192 283 131. A technique called molecular combing has been applied to study DNA replication. Replicating DNA is differentially labeled at successive time points after the beginning of DNA synthesis, then the DNA is extracted and combed on a glass surface,

Some molecular combing procedures involve the use of mapping probes, such as those described and incorporated by reference to Bensimon, et at, U.S. Pat. No. 6,248,537 for example to identify particular genetic loci in combed genomic DNA. Such mapping probes and procedures are unnecessary for parameters of DNA replication such as replication fork speed, inter-origin distance as whole genome information. However, when one would like to focus analysis of DNA replication on certain loci of the genome or localization origins of DNA replication in or around such loci, such mapping probes and procedures may be combined with the procedures described herein for DNA replication labelling.

In these methods, the;detected signals appear as linear fluorescent signals, which result from intermediates produced, by incorporation of nucleotides tagged with different colored dyes during DNA replication. In situations where analysis of DNA replication is focused on certain loci, additional signals from labeled probes hybridized to the, replicating or replicated DNA in or around the loci of interest may also be detected and analyzed.

Control sequence. A control sequence is any sequence furnished by a genetic database or produced by another method (usually by a method other than, molecular combing) that can be compared to a GMC sequence obtained by molecular combing. In many instances, the control sequence will be one stored as data in a database for which a deduced or theoretical GMC is produced for later comparison with a test data obtained probing on a test genomic DNA region with the same GMC in conjunction with molecular combing. Two or more different GMC patterns obtained by molecular combing may also be compared, a subpart of them being designated reference or control patterns. In the example given below on biomarker identification, control patterns are defined as GMC patterns associated to individuals which have been characterized as having 2 or less SMN1 copies with other quantification techniques.

Complex regions of the genome. A complex genetic region is a region that contains segmental duplications, a high number of repeat elements, microsatellites or all together, that prevent accurate sequencing assembly of the region. Other regions containing segmental duplications are for example regions associated to Gaucher disease, fascio-scapulo-humeral muscular dystrophy or azoosperima. Further description of complex regions is provided at and incorporated by reference to Bailey, et al, Science 297, 1003 (2002) or to;

https://_hocking.biology.ualberta.ca/courses/genet302/uploads/winter08/Gen302%20Rea dings/22/22%20SUPPLEMENTAL%20SheEichler%20-%20Shotgun%20sequence%20assembly%20and%20recent%20segmental%20duplications%20w ithm%20the%20human%20genome.pdf (last accessed Oct. 12,2017).

Spinal Muscular Atrophy. Spinal muscular atrophy (SMA), also called autosomal recessive proximal spinal muscular atrophy and 5q spinal muscular atrophy in order to distinguish it from other conditions with similar names, is a rare neuromuscular disorder characterised by loss of motor neurons and progressive muscle wasting, often leading to early death. The disorder is caused by a genetic defect in the SMN1 gene, which encodes SMN, a protein widely expressed in all eukaryotic cells and necessary for surv ival of motor neurons. Lower levels of the protein results in loss of function of neuronal cells in the anterior horn of the spinal cord and subsequent system-wide atrophy of skeletal muscles. Spinal muscular atrophy manifests in various degrees of severity, which all have in common progressive muscle wasting and mobility impairment. Proximal muscles and respiratory muscles are affected first. Other body systems may be affected as well, particularly in early-onset forms of the disorder. SMA is the most common genetic cause of infant death. Spinal muscular atrophy is an inherited disorder and is passed on;in an autosomal recessive manner, in December 2016, nusinersen became the first approved drug to treat SMA while several other compounds remain in clinical trials.

SMN1 is the telomeric copy of the gene encoding the SMN protein; the centromeric copy is termed SMN2. SMN1 and SMN2 are part of a 500 kb inverted duplication on chromosome 5q13. This duplicated region contains at least four genes and repetitive elements which make it prone to rearrangements and deletions. The repetitiveness and complexity of the sequence have also caused difficulty in determining the organization of this genomic region. SMN1 and SMN2 are, nearly identical and encode the same protein. The critical sequence difference between the two is a single nucleotide in exon 7 which is thought to be an exon splice enhancer. It is thought that gene conversion events may involve the two genes, leading to varying copy numbers of each gene. Mutations in SMN1 are associated with spinal muscular atrophy. Mutations in SMN2 alone do not lead to disease, although mutations in both SMN1 and SMN2 result in embryonic death.

### EMBODIMENTS

The following embodiments directed to specific aspects of the invention are intended to further illustrate certain steps and combinations of steps associated with the method disclosed herein and are not intended to limit the scope of the claims.

Embodiment 1. A method for detecting genomic DNA arrangement associated with a genetic disease, disorder or condition comprising

producing or providing a set of labelled probes covering a genomic region of interest that contains a gene of interest associated with the genetic disease, disorder or condition,

hybridizing the labelled probes to said region, wherein said probes are labelled with one of several different colors, wherein each color designates a different target or class of target sequences;

detecting a hybridization pattern formed on the genomic region of interest, and

reconstructing the hybridization patterns for each allele on the genomic region of interest;

comparing the hybridization pattern of the labelled probes on the genomic region of interest between individuals in order to identify genetic direct or indirect biomarkers for the presence of carrier for the disease, disorder or condition.

Embodiment 2. The method of embodiment 1, wherein the genetic disease, disorder, or condition is spinal muscular atrophy (“SMA”) and the region of interest is an SMA locus,

Embodiment 3. The method of embodiment 2, wherein the labelled probes contain a color-coded probe that specifically recognizes SMN genes present in the control genomic DNA sequence which is a GRCh38/hg38 assembly or another control sequence spanning the SMA locus,

Embodiment 4. The method of embodiment 3, wherein the labelled probes further comprise bacterial artificial chromosome (BAC) or other orienting probes that when bound to the genomic region of interest orientate it with respect to a chromosomal centromere and telomere.

Embodiment 5. The method of embodiment 4, wherein the labelled probes further comprise probes that bind to repeat regions or other segments of the genomic region of interest.

Embodiment 6. The method of embodiment 5, wherein the genomic region of interest is obtained from a subject who has SMA, is a carrier of SMA, or who is otherwise at risk of having or carrying SMA.

Embodiment 7. The method of embodiment 6, wherein the genomic region of interest is obtained from germ cells, ovum, or sperm.

Embodiment 8. The method of embodiment 6, wherein the genomic region of interest is obtained in utero.

Embodiment 9. The method of embodiment 6, wherein the genomic region of interest is obtained in from a prospective parent.

Embodiment 10. The method of embodiment 6, wherein the genomic region of interest is obtained from a subject having an African or African-American genetic profile.

Embodiment 11. The method of embodiment 6, further comprising diagnosing, counseling or treating a subject a subject who has SMA, is a carder of SMA, or who is otherwise at risk of having or carrying SMA.

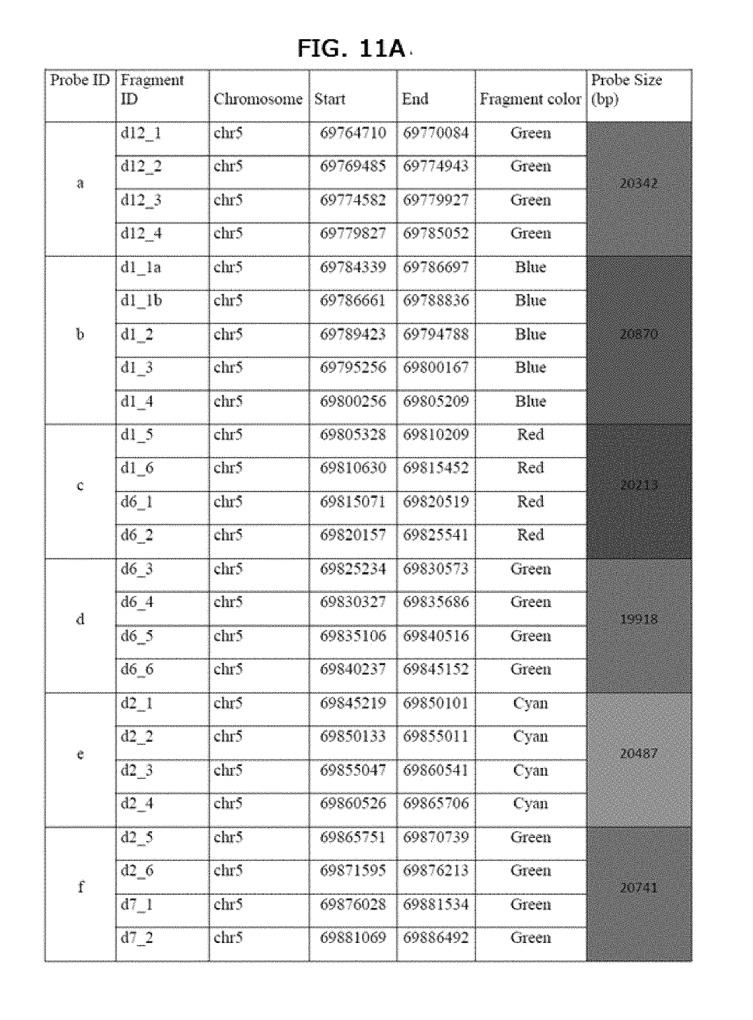

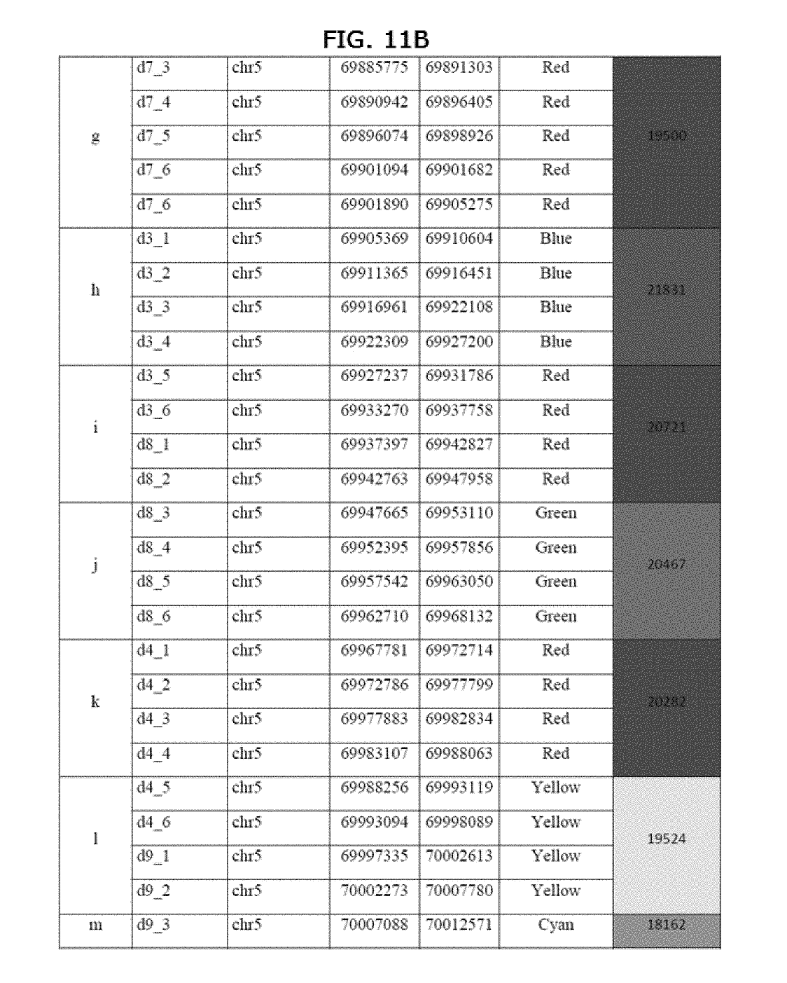

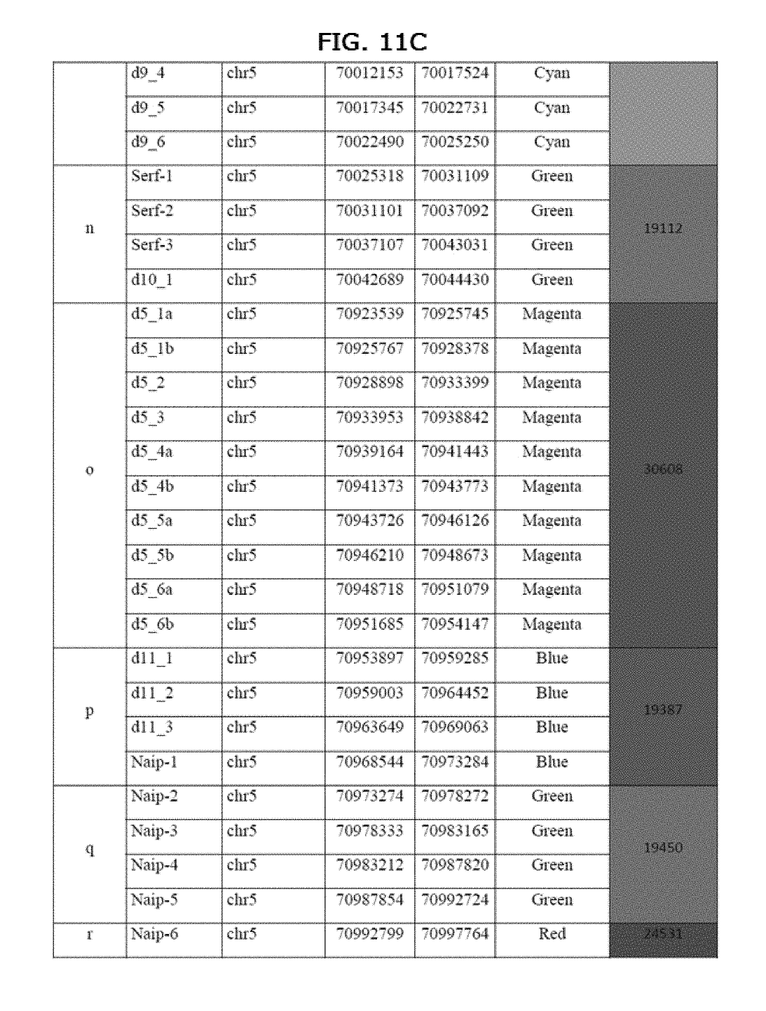

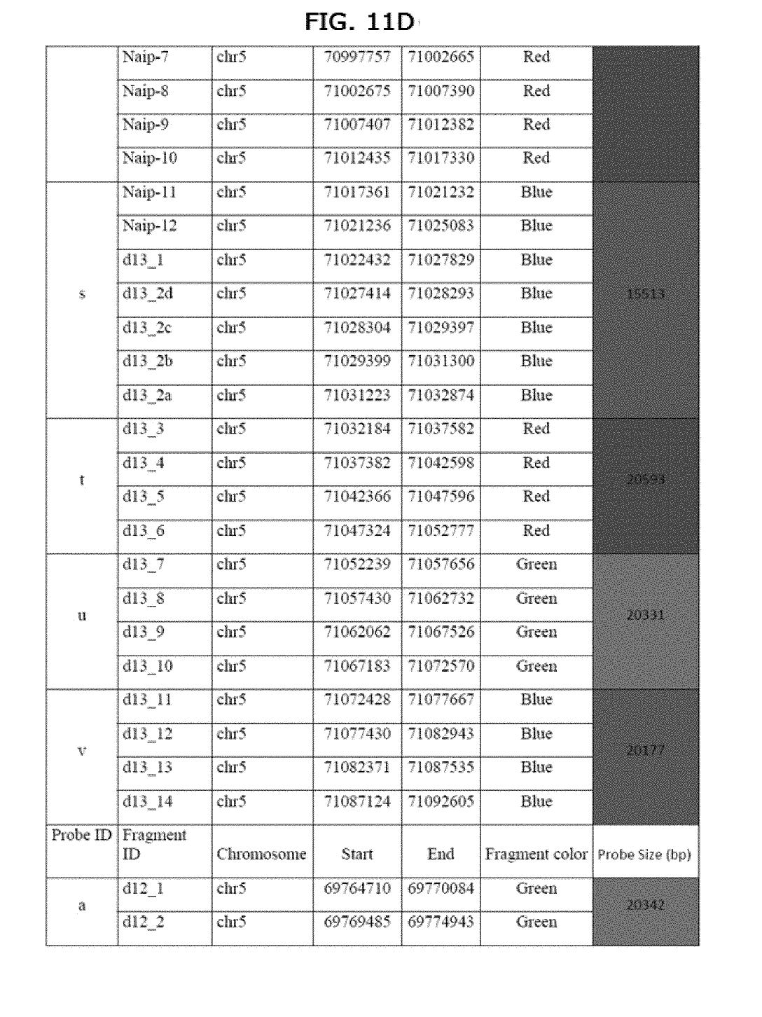

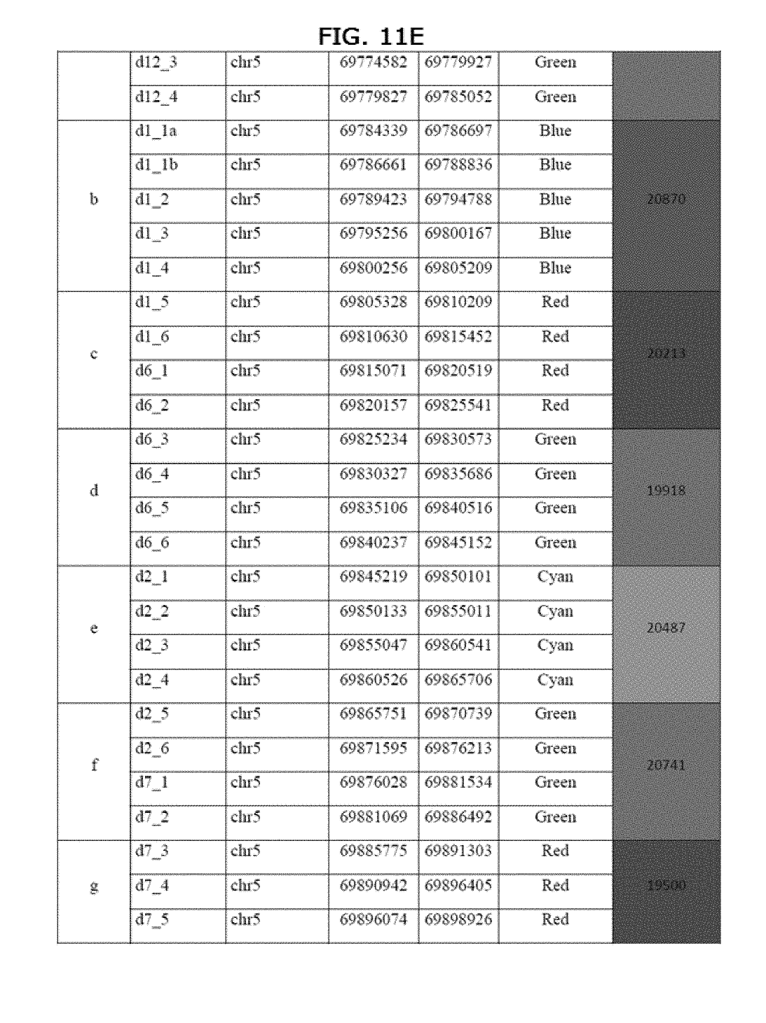

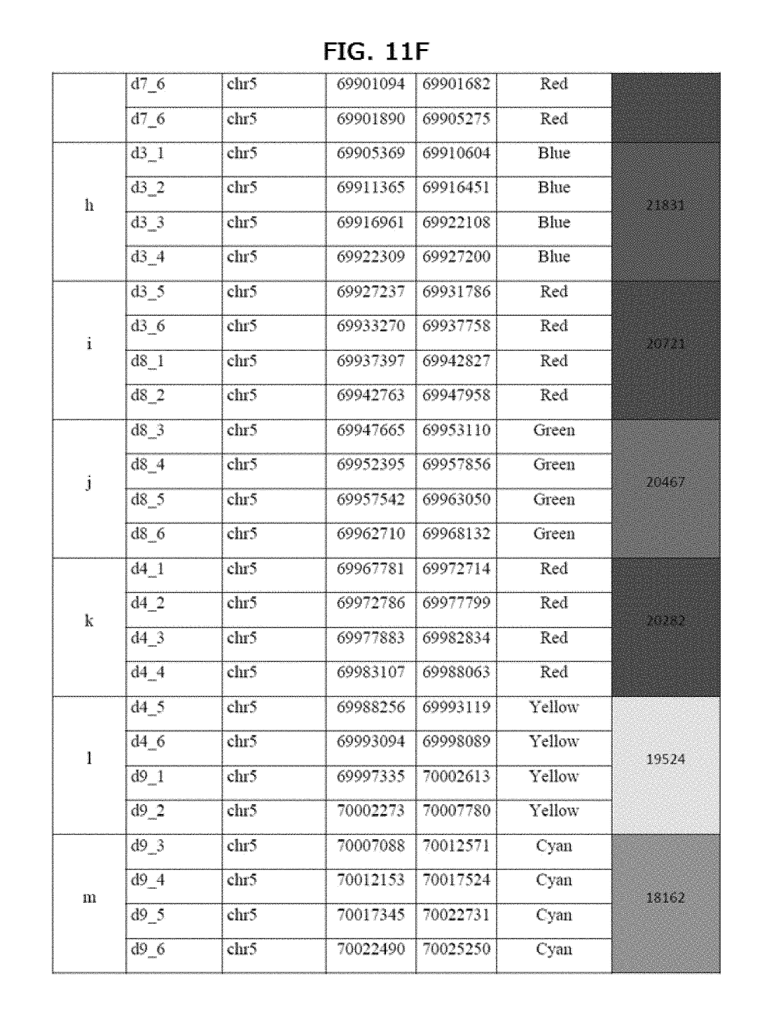

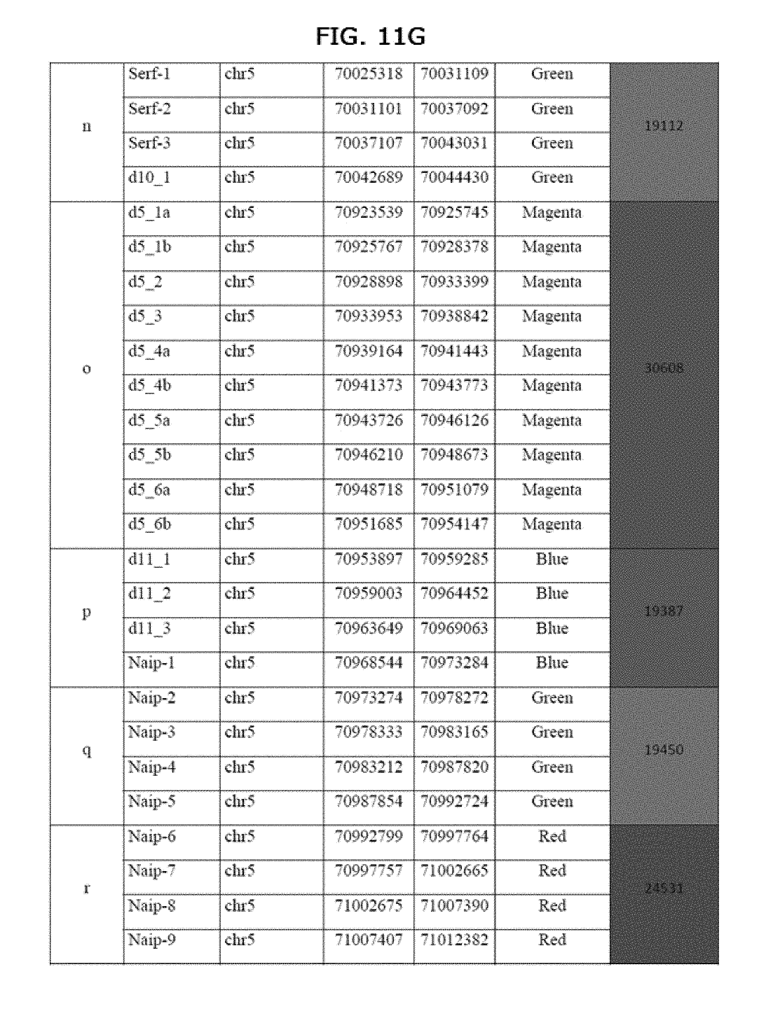

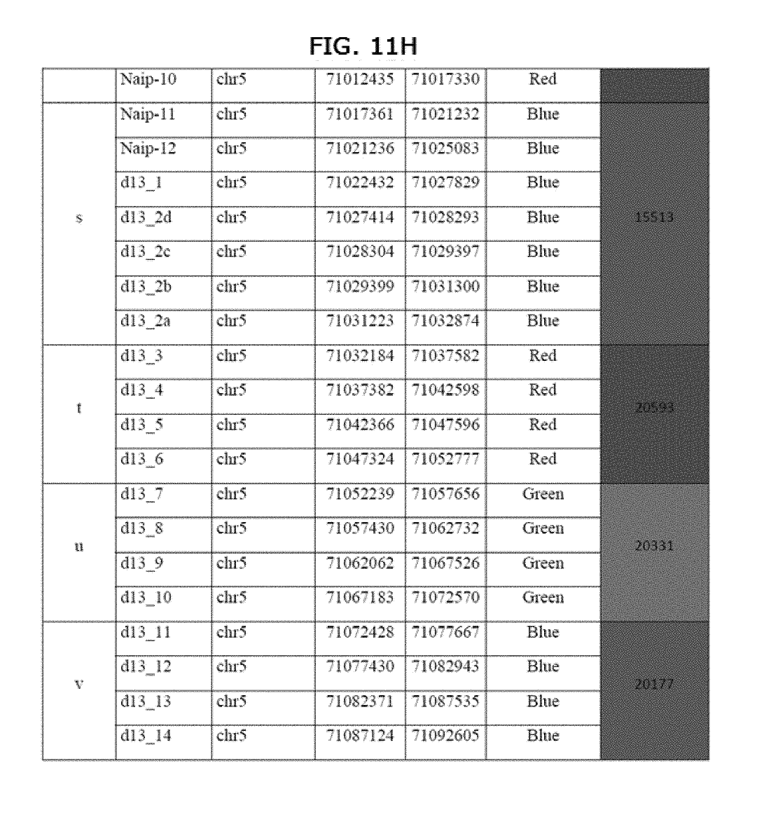

Embodiment 12. A composition comprising a set Genomic Morse Code (“GMC”) probes suitable for detecting and mapping SMN genes in a genomic DNA region of interest. Various combinations of the sets of GMC probes or all the probes described in FIG. 11 (Table A) may be selected for use in the claimed molecular combing method, for example, a more limited set of probes may be selected to focus the analysis on a particular subsegment of the SMA region. Those skilled in the art equipped with the identity of the probes disclosed herein may select a suitable set of 2, 10, 20, 30, 40, 50, 60, 70, 80, 90, 100 or more probes for use. One or more colors or types of probes described by FIG. 11 may be included or omitted.

Embodiment 13. A kit comprising a set of labelled probes suitable for detecting and mapping SMN genes, a control genomic DNA sample or providing a deduced or theoretical GMC pattern of a control DNA sample, instructions for use and packaging materials.

Embodiment 14. A method for characterizing at least one allele in a complex genetic region comprising:

selecting a genetic segment of interest,

producing or providing a set of labelled probes covering the genomic region of interest that contains an allele of interest,

hybridizing the labelled probes to said region, wherein said probes are labelled with one of several different colors, wherein each color designates a different target or class of target sequences;

detecting a hybridization pattern formed on the genomic region of interest, and

reconstructing the hybridization patterns for each allele on the genomic region of interest;

comparing the hybridization, pattern of the labelled probes on the genomic region of interest with a control hybridization pattern.

Embodiment 15. The method of embodiment 14, wherein the at least one allele is associated with a genetic disease, disorder or condition.

Embodiment 16, The method of embodiment 14, further comprising identifying at least one genetic biomarker for one or more alleles in the region of interest that distinguishes it from the corresponding region of interest in the control sequence.

Embodiment 17. The method of embodiment 16, wherein the biomarker identifies a cis duplication of SMN1.

Embodiment 18. A method for discovering an error in a sequence of a genomic region of interest described in a database comprising:

selecting a genetic segment of interest,

producing or providing a set of labelled probes covering the genomic region of interest that contains a segment to be inspected for errors,

hybridizing the labelled probes to said region, wherein said probes are labelled with one of several different colors, wherein each color designates a different target or class of target sequences;

detecting a hybridization pattern formed on the genomic region of interest, and

comparing the hybridization pattern of the labelled probes on the genomic region of interest with a theoretical hybridization pattern deduced from the database sequence to be inspected for errors;

identifying an error when a discrepancy is detected between the hybridization pattern of the genomic region of interest and the deduced hybridization pattern for the genomic region of interest from the database.

Embodiment 19. A method for identifying unpublished copy number variations (“CNVs”) in a sequence of a genomic region of interest comprising:

selecting a genetic segment of interest from genomic DNA to be tested for presence of copy number variations (“CNVs”);

producing or providing a set of labelled probes covering the genomic region of interest,

hybridizing the labelled probes to said region of interest, wherein said probes are labelled with one of several different colors, wherein each color designates a different target or class of target sequences;

detecting a hybridization pattern formed on the genomic region of interest, and

comparing the hybridization pattern of the labelled probes on the genomic region of interest with a theoretical hybridization pattern deduced from a control database sequence to be used as a referent for identifying unpublished CNVs; and

identifying a new CNV when a copy number of a particular segment of the region of interest differs from that a referent hybridization pattern.

Embodiment 20. The method of embodiment 19, wherein the referent hybridization pattern is deduced from a known genomic DNA sequence.

Embodiment 21. The method of embodiment 19, wherein the identified CNV is in the SMA genomic region.

Embodiment 22. The method of embodiments 14-19, where the biomarkers found are selected fragments of SMA region composed of combinations of complete or partial duplications of Genomic Morse Code (“GMC”) probes on SMA region

In some embodiments including any of those described above an entire or partial set of probes described by FIG. 11 (Table A) may be used to probe a genomic DNA sequence such as the SMA region or their corresponding sequences used to produce a theoretical GMC for a control or database sequence. A combination or probes will be selected to identify target segments of a genomic region of interest or omit probes that identify segments of the genomic region that are not of interest.

The following examples are intended to further illustrate certain steps and combinations of steps associated with the method disclosed herein and are not intended to limit the scope of the claims.

### EXAMPLES

### SMA Genomic Morse Code

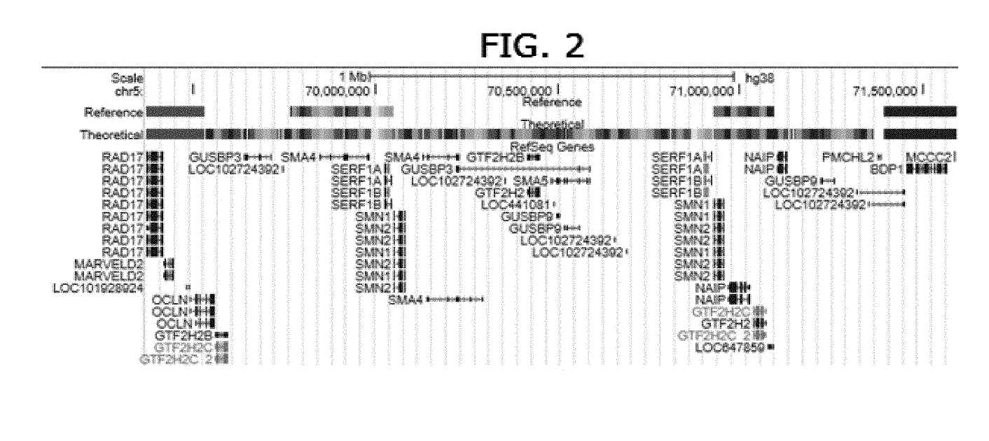

A specific Genomic Morse Code (GMC) was developed to cover the region of interest as it is described in the reference human genome database GRCh38/hg38 (https://_genome.ucsc.edu/); see FIG. 2 which depicts relative positions of GMC DNA probes according to GRCh38/hg38 human genome assembly (http://_genome.ucsc.edu/). Due to the high segmental duplication of this region, the DNA probes designed hybridize as fragments along the region to form the theoretical GMC. More than 2 Mb of the SMA locus are thus covered.

FIG. 2 shows die Genetic Morse Code (GMC) with relative positions of DNA probes according to GRCh38/hg38 human genome assembly (http://genome.ucsc.edu/). The relative position of genes localized on the SMA locus are indicated below the GMC SMN1 and SMN2 genes are covered by a unique magenta probe. Two BACs are used as flanking probes to orientate the SMA locus: one SMN gene associated with the centromeric red probe (SMN_C), and one SMN with the telomeric blue probe. Top. “Reference” Position of the reference DNA fragments designed on the SMA locus. Bottom: “Theoretical” Color-coded pattern form by hybridization of the probes along the SMA locus. (GRCh38/hg38; chr5: 69071065-71594127).

The coordinates of the probes relative to the human GRCh38/hg38 sequence (chr5: 69,764,710-71,092,605) are listed in table A. Probe size ranges from 18,162 to 30,608 bp in this example. The coordinates in Table A (a color version of which appears as FIGS. 12A-12H) correspond to human GRCh38/hg38 sequence.

The probe fragments were produced either by long-range PCR using LR Taq DNA polymerase (Roche, kit code: 11681842001) or by direct gene synthesis (GeneCust, Dudelange, Luxemburg). The anchoring blue and red probes correspond to Bacterial Artificial Chromosomes (BAC) RP11-427A10 and RP11-350A19 (Invitrogen), respectively. PCR products were ligated in the pCR-XL-TOPO® vector using the TOPO® XL PCR cloning Kit (Invitrogen, France, code K455010). The two extremities of each fragment were sequenced for verification purpose.

### Analysis of SMA Fluorescent Arrays and SMA Locus Reconstitution

The GMC described in the Example was hybridized on combed genomic DNA extracted from amniocyte-derived cell cultures from forty eight African-American individuals.

The fluorescent signals obtained on those samples are compared to the theoretical GMC deduced from the human reference database (GRCh38/hg38),

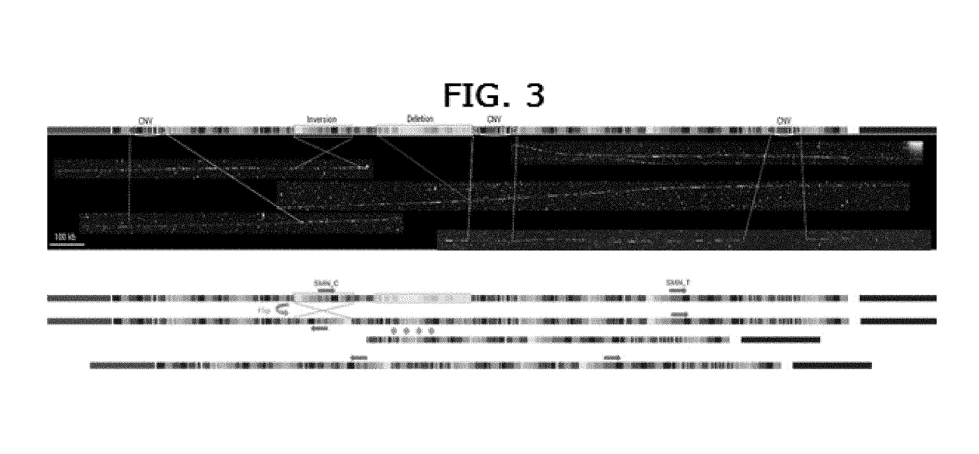

FIG. 3 shows the alignment of SMA fluorescent, arrays with the theoretical GMC and SMA locus reconstitutions. This alignment of the different color-patterns revealed some discrepancies which are described by FIG. 3 including:
an inversion of the centromeric SIN copy suggesting a different orientation of the region covering the SMN_C gene. The SMN genes are in a tail to-tail orientation and not in a head-to-tail orientation as annotated in the reference sequence (magenta arrows), and
a deletion of a color-pattern suggesting an absence of this DNA sequence in samples analyzed

### CNV Identification

Another discrepancy that was observed between fluorescent signals using molecular combing and theoretical GMC deduced from the human reference database (GRCh38/hg38) was a repeat sequence made of alternating red and blue segments with a variable number of repeated units, localized along the SMA locus indicating the presence of copy number variation sequence. The CNV discovered is currently unpublished, i.e. it has never been mentioned in any scientific publication about SMA or in any database containing SMA genomic information. The size of the CNV is estimated between 15 to 30 kb and has been identified as composed of all or subparts of the GMC fragments shown in Table B.

TABLE B
List of fragments covering the CNV sequence
(Coordinates correspond to GRCh38/hg438 human reference database)

| Probe | Fragment | | | | Fragment |
|---|---|---|---|---|---|
| ID | ID | Chromosome | Start | End | color |
| g | d7_3 | chr5 | 69885775 | 69891303 | Red |
| | d7_4 | chr5 | 69890942 | 69896405 | Red |
| | d7_5 | chr5 | 69896074 | 69898926 | Red |
| | d7_6 | chr5 | 69901094 | 69901682 | Red |
| | d7_6 | chr5 | 69901890 | 69905275 | Red |
| h | d3_1 | chr5 | 69905369 | 69910604 | Blue |
| | d3_2 | chr5 | 69911365 | 69916451 | Blue |
| | d3_3 | chr5 | 69916961 | 69922108 | Blue |
| | d3_4 | chr5 | 69922309 | 69927200 | Blue |
| i | d3_5 | chr5 | 69927237 | 69931786 | Red |
| | d3_6 | chr5 | 69933270 | 69937758 | Red |

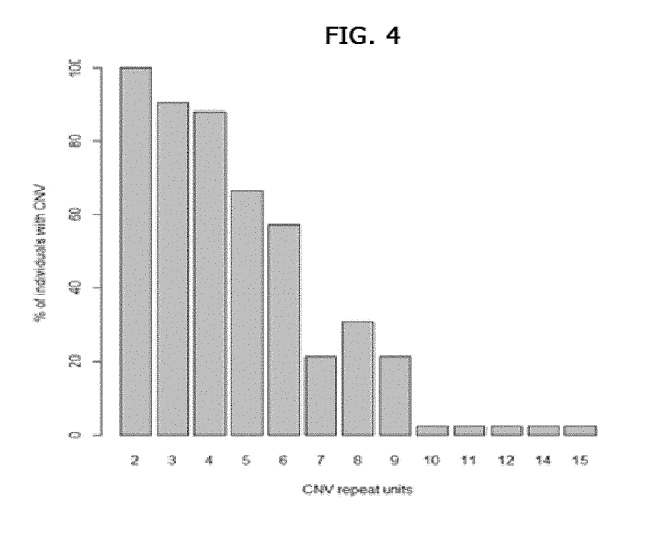

The analysis of the CNV reveals a high variability in term of number of repeated units count. Applied on the 48 individuals processed in Example 1 (see below), the number of repeated units ranges from 2 to 15 as presented in the histogram below (FIG. 4).

However, the identification of the CNV according to the number of repeated units associated with the color-coded pattern created by the GMC allows to map the alleles of some individuals from molecular combing data. The CNVs are identified on molecular combing data by the number of repeated units of blue and red probes associated with the color-coded pattern created by the GMC described above (as seen in FIG. 3),

### Haplotyping

The observation of CNVs in molecular combing signals showed us variability of CNV lengths as well as CNV positions along the SMA region, not only between individuals but also between alleles of the same DNA. Consequently, the molecular combing signals contain information that enables us to reconstruct haplotype phasing of individuals with high certainty. The inventors present here two different processes to reconstruct alleles of an individual, one automated and one manual.

### 1. Automated Allelic Reconstruction

These are defined as a process in order to reconstruct alleles from molecular combing signals in the case where the anchoring probes of each extremity of the genetic region of interest are unambiguously identifiable in available data. For example, the red and blue anchoring probes defining the centromeric and telomeric extremities of the SMA region are easily distinguishable from probes within the region due to their lengths of 1 and 199 kb, respectively.

The different steps of the allele reconstruction method are the following;
Compute a distance value between each pair of combing signals. The distance value must reproduce the level of quasi-perfect overlap between signals in terms of orientation of color and length information contained in each signal, Usual distances can be used, as well as customized distances specifically adapted to the characteristics of the region of interest.
Create all possible pathways going from signals containing centromeric anchoring probe to signals containing telomeric anchoring probe using the distance matrix computed before and a distance threshold. Attribute a complexity score based on length and presence of pattern multiple occurrences for each pathway.
Cluster pathways using a distance function that can be usual or customized.
Compute coverage of each pathway using a distance function between each signal of the data set and the pathway. The distance function can be usual or customized.
Compute for each pair of pathways a confidence score based on combined pathway coverages and complexity scores. The confidence score decreases with decreasing, pathway coverage or increasing complexity scores.
Select pairs of pathways that have, the best confidence score.

### 2. Manual Allelic Reconstruction

The inventors present here a manual method for allele reconstruction that was used specifically for signals hybridized with SMA GMC v3.
Gather all signals containing the centromeric anchoring probe and identify two groups with each a different color-code pattern when possible.
Do the same with signals containing the telomeric anchoring probe
Gather signals with magenta probe (SMN probe) and identify all the different color-code patterns around the magenta probe (usually defined by orientation of yellow and magenta probes, as well as lengths of, neighboring (CNVs)
Assemble all identified groups into 2 distinct complete alleles based on overlapping of color-coded patterns at the extremities of each different group.

Despite the complexity of the SMA region in terms of genetic duplication and variability, the reconstruction of SMA alleles is possible using molecular combing data due to allelic genetic variability and frequent occurrence of long signals ranging from 500 kb up 1 kb. Examples of allelic reconstruction are disclosed below.

### Identification of Biomarkers for Cis-Duplication of SMN1

With all tools mentioned before, it is possible to identify presence of unknown allelic large rearrangements in a population. In the application case of SMA region analysis, molecular combing can be used to identify ethnic-specific biomarkers for the presence of SMN1 cis-duplication in an allele in a population.

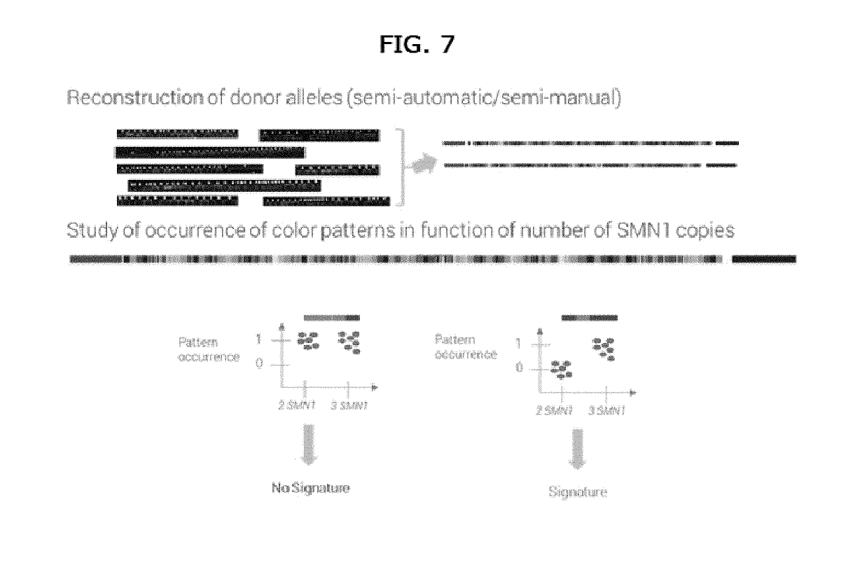

The data analysis method is based on comparison of color pattern occurrences in reconstructed alleles between combing data obtained on a “group control” composed of individuals without SMN1 cis-duplication and on a “test control” composed of individuals with SMN1 cis-duplication; see FIG. 7. ROC curve, analysis is then applied to determine the diagnostic performance of color patterns of different lengths for specificity and sensitivity to differentiate the two groups.

The section below presents the biomarkers identified with this methodology on a data set of 48 DNA samples from African-American individuals.

### Biomarker Identification for Cis-SMN1 Duplication on 48 African-American Individuals with Different MLPA Quantifications of SMN1 and SMN2

We applied the method described above to 48 African-American individuals, separated into two different groups in function of their quantification of SMN1 genes.

Preparation of embedded DNA plugs from amniocyte-derived cell cultures. Agarose plugs with embedded DNA from African-American amniocyte-derived cell cultures are prepared as described in Schurra and Bensimon (Schurra and Bensimon 2009). Briefly, cells were resuspended in Trypsine/PBS (1:1) at a concentration of 10⁶ cells 45 μL mixed thoroughly at a 1:1 ratio with a 1.2% w/v solution of low-melting point agarose (Nusieve GTG, ref. 50081, Cambrex) prepared in 1× PBS at 50° C. 90 μL of the cell/agarose mix was poured in a plug-forming well (BioRad, ref 170-3713) and left to cool down at least 30 min at 4° C. Agarose plugs were incubated overnight at 50° C. in 250 μL of a 0.5M EDTA (pH 8), 1% Sarkosyl, 2 μg/μL proteinase K (Eurobio, code: GEXPRK01, France) solution, then washed three times in a Tris 10 mM, EDTA 1 mM solution for 30 min at room temperature

Final extraction of DNA and Molecular Combing. Plugs of embedded DNA from amniocyte-derived cell cultures were treated for combing DNA as previously described (Schurra and Bensimon 2009). Briefly, plugs were melted at 68° C. in a MES 0.5 M (pH 5.5) solution for 20 min, and 1.5 units of beta-agarase (New England Biolabs, ref. M0392S, MA, USA) was added and left to incubate for up to 16 h at 42° C. The DNA solution was then poured in a Disposable DNA reservoir (Genomic Vision S.A., Paris, France) and Molecular Combing was performed using, the Molecular Combing System (Genomic Vision S.A., Paris, France) and CombiCoverslips® (20 mm×20 mm, Genomic Vision S.A., Paris, France). The combed surfaces were dried for 4 hours at 60° C.

Labelling of SMA probes. The coordinates of the probes relative to the human GRCh38/hg38 sequence (chr5: 69,764,710-71,092,605) are listed in Table A above. For labelling, the SMA GMC v3 probes are grouped according to the incorporated hapten: probe fragments associated to the color blue in Table A are jointly labelled with 3-Amino-3-Deoxydigoxigenin-9-dCTP (AminoDIG-9-dCTP); those associated to color green are jointly labelled with Fluorescein-12-dUTP (Fluo-dUTP); those associated to color red are jointly labelled with biotin-11-dCTP (Biot-dCTP). Moreover, probe fragments associated to the color cyan in Table A are jointly co-labelled with both AminoDIG-9-dCTP and Fluo-dUTP; those associated to color magenta are jointly co-labelled with both AminoDIG-9-dCTP and Biot-dCTP; those associated to color yellow are jointly co-labelled with both Fluo-dUTP and Biot-dCTP. 200 ng of each BRCA probe group were labelled using conventional random priming protocols with the BioPrime® DNA kit (Invitrogen, code: 18094-011, CA, USA) according to the manufacturer's instructions except the dNTP mix from the kit was replaced by the mix specified in Table C and the labelling reaction was allowed to proceed overnight. After labelling, labelled product is purified with PureLink® PCR Purification Kit (ThermoFischer Scientific; Code K310001) according to the manufacturer's instructions.

TABLE C
dNTP mix used for probe labelling of SMA GMC v3

| | Non-modified dNTPs | |
|---|---|---|
| | Invitrogen, ref. | |
| Labelling | 10297-018) | Hapten-coupled dNTP |
| Fluo-dUTP | dATP, dCTP, dGTP | Fluorescein-12-dUTP 20 μM |
| Green | 40 μM each | (Sigma Aldrich, code |
| | dTTP 20 μM | 000000011373242910) |
| AminoDIG- | dATP, dTTP, dGTP | 3-Amino-3-Deoxydigoxigenin- |
| 9-dCTP | 40 μM each | 9-dCTP 20 μM (Perkin Elmer, |
| Blue | dCTP 20 μM | code NEL562001EA) |
| Biot-dCTP | dATP, dTTP, dGTP | Biotin-11-dCTP 20 μM Perkin |
| Red | 40 μM each | Elmer code NEL538001EA) |
| | dCTP 20 μM | |

Hybridization of SMA GMC v3 on combed genomic DNA and detection. Subsequent steps were also performed essentially as previously described in Schurra and Bensimon, 2009 (Schurra and Bensimon 2009). Briefly, a mix of labelled probes (250 ng of each probe) were ethanol-precipitated together with 10 μg herring sperm DNA and 2.5 μg Human Cot-1 DNA (Invitrogen, ref. 15279-011, CA, USA), resuspended in 20 μL of hybridization buffer (50% formamide, 2× SSC. 0.5% SDS, 0.5 Sarkosyl, 10 mM NaCl, 30% Block-aid (Invitrogen, ref. B-10710, CA, USA). The probe solution and probes were heat-denatured together on the Hybridizer (Dako, ref. S2451) at 90° C. for 5 mm and hybridization was left to proceed on the Hybridizer overnight at 37° C. Slides were washed 3 times in 60° C. pre-warmed 2× SSC solution for 5 min at room temperature. After the last washing steps, the hybridized coverslips were gradually dehydrated in 70%, 90% and 100% ethanol solution and air dried. For detection, 20 μL of the antibody solution diluted in Block-Aid® was added on the slide and covered with a combed coverslip and the slide was incubated in humid atmosphere at 37 for 20 min. Detection of the GMC SMA v3 was carried Out using a Alexa Fluor® 647-coupled mouse monoclonal anti-digoxygenin (Jackson Immunoresearch, code 200-162-037) antibody in a 1:25 dilution for AminoDIG9-dCTP-labelled probes, a Cy3-coupled mouse monoclonal anti-Fluorescein (Jackson Immunoresearch, code 200-602-156) antibody in a 1:25 dilution for Fluo-dUTP-labelled probes and an BV480-coupled streptavidin (BD Biosciences, code 564876) in a 1:25 dilution for Biot-dCTP-labelled probes. The slides were then washed 3 times in a 2× SSC, 1% Tween 20 solution for 3 min at room temperature and all glass coverslips were dehydrated in ethanol and air dried.

Analysis of SMA detected signals and allelic characterization. Hybridized-combed DNA from amniocyte-derived cell cultures preparation were scanned without any mounting medium using an inverted automated epifluorescence microscope, equipped with a 40× objective (FiberVision®, Genomic Vision S.A., Paris, France) and the signals were analysed by an in house software (FiberStudio® BRCA, Genomic Vision S.A., Paris, France).

Signals were detected on scanned images using both a detection algorithm implemented in house software (U.S. 62/306,296) and manual detection. Alleles were reconstituted using both automatic and manual methods described above.

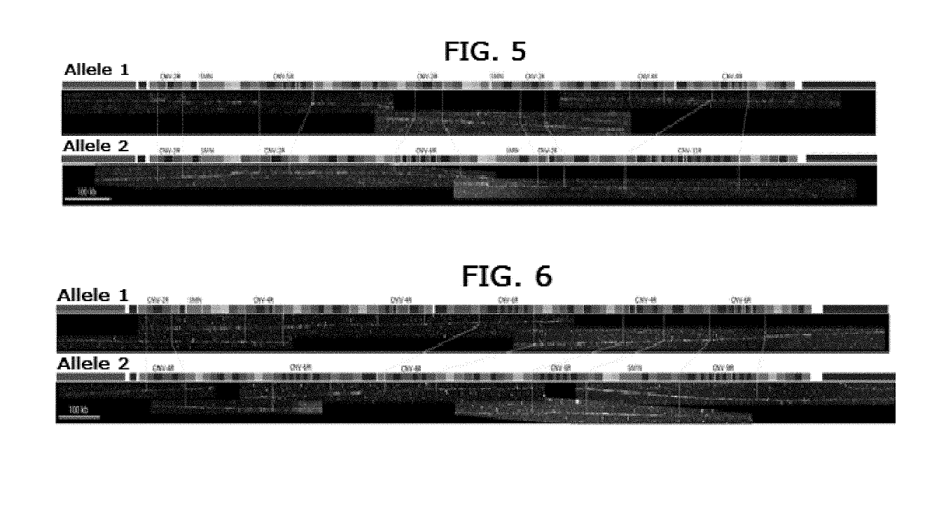

As shown in FIG. 5, the 2 alleles of one of the DNA samples of the study were reconstituted. This particular sample was characterized as having 2 SMN1 and 2 SMN2 copies by MLPA [Huang 2007]. Each allele contains a centromeric and a telomeric copy of the SMN gene as shown by the presence of the magenta probes. Moreover, the SMA GMC hybridization revealed that the genomic organization surrounding each copy differs by the number of CNV repeat units. The centromeric SMN gene of the allele 1 is surrounded by two CNV with 2 and 5 copies, while the same region on the allele 2 carries CNV with only 2 copies,

FIG. 6 shows allelic reconstitution performed on another of the 48 DNA of this study, this sample having only 2 SMN1 copies and no SMN2 gene according to MLPA characterization [Huang 2007]. The allele reconstitution allows to map the two SMN1 genes in the two different alleles confirming that this individual is a non-carrier. As we found in the previous analysis done on the individual with 2 SMN1 and 2 SMN2 copies, the CNV presents in this region is highly variable with a number of repeated sequence from 2 to 9, In addition, the number of CNV also varies, 6 CNV's were found on allele 1 while only 5 on allele 2.

Biomarkers identification for cis-duplication of SMN1. Each, of the 48 samples from African-American individuals were processed by MLPA [Huang 2007] in order to quantify the number of SMN1 and SMN2 present in each DNA. Seven individuals were excluded from biomarker identification analysis due to discrepancies between SMN quantification using MLPA and SMN quantification using combing (i.e., the number of magenta probes present in reconstructed alleles). The final data set used was composed of a control group containing 23 individuals with at most 2 SMN1 copies and a test group containing 18 individuals containing 18 individuals with at least 3 SMN1 copies.

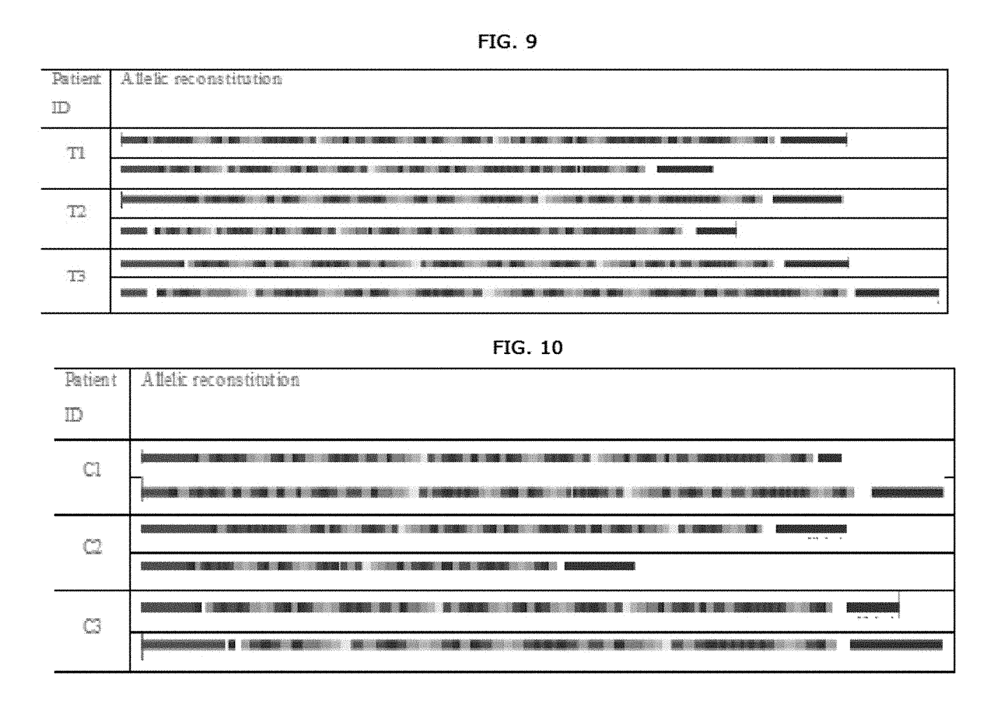

FIG. 9 and FIG. 10 present example allelic reconstitutions for individuals from Test and Control groups, respectively. The analysis of biomarker identification was performed on data of reconstructed alleles for every individual with the following rules:
Each allele was considered as an ordered sequence of probes, each probe having one of 8 possible value (anchoring red, anchoring blue, red, blue, green, cyan, magenta, yellow).
All patterns of size from 2 to 30 probes were evaluated
An independent analysis was performed for each pattern
The diagnosis performance i.e. ability to distinguish individuals of control group from individuals of test group) of each pattern were defined by the sum of sensitivity and specificity compound.

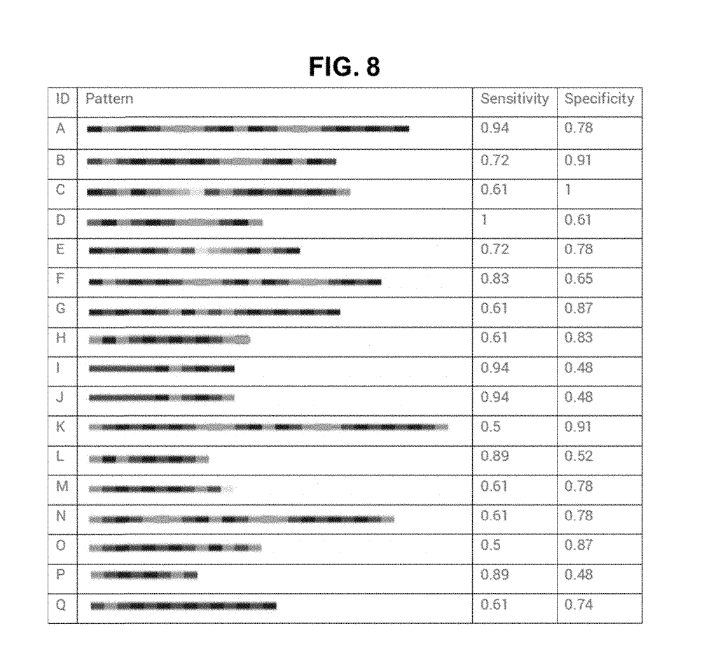

A set of 17 subregions of the SMA genetic region have been identified as being pertinent, either individually or in combination with one another, to distinguish between test group and control group individuals (see FIG. 8). Consequently, these subregions are good candidates for biomarkers of the presence of the cis-duplication of SMN1, either individually or in combinations with one another. For example, we observe that pattern A from FIG. 8 is present in allelic reconstitutions of the 3 patients from test group presented in FIG. 9 whereas it cannot be found in the 3 patients from control group presented in FIG. 10.

These patterns range in size from 140 kb to 54 kb. Each of them can be described as a sequence of smaller genetic elements (from 40 kb to 200 kb) that are frequent along the SMA region. The presence of smaller genetic elements, when studied independently, does not bring any information on the presence of SMN gene duplication on the same allele. However, the geographical positioning of those elements relatively to one another is the critical information that defines the biomarker of the cis-duplication of SMN1.

Based on these results, a more detailed analysis of these probe patterns can be performed, either using specifically adapted GMC with molecular combing, or using other techniques such as sequencing or qPCR, to further characterize the sequences of the found biomarkers. However, we extrapolated the probe pattern for some of the potential biomarkers based on their color sequence. Table D show the probe composition of pattern J from FIG. 8 and Table E the probe composition of pattern A.

TABLE D
List of coordinates tbr probes of pattern J from FIG. 8. The probes are
composed of combinations of complete or partial duplications of fragments of the GMC defined
in FIG. 2. The last column specifies which fragments of FIG. 2 compose each probe of pattern J.
Coordinates correspond to GRCh38/hg38 human reference database.

| Probe ID | Chromosome | Start | End | Probe color | Original Fragments |
|---|---|---|---|---|---|
| RP11-427A10 | chr5 | 69371065 | 69531487 | Red | RP11-427A10 BAC |
| d13_dup | chr5 | 69535271 | 69555469 | Blue | d13_11, d13_12, d13_13 and d13_14 |
| d13_dup_2 | chr5 | 69555440 | 69574739 | Green | d13_7, d13_8, d13_9 and d13_10 |
| d13_dup_3 | chr5 | 69575107 | 69595239 | Red | d13_3, d13_4, d13_5 and d13_6 |
| d13_Naip_dup | chr5 | 69595660 | 69610510 | Blue | d13_1, d13_2a, d13_2b, d13_2c, |
| | | | | | d13_d, Naip_11, Naip_12 |
| Naip_dup | chr5 | 69610541 | 69628397 | Red | Naip_6, Naip_7, Naip_8, Naip_9, |
| | | | | | Naip_10 |
| d2_d7_dup | chr5 | 69666696 | 69685929 | Green | d7_1, d7_2, d2_5, d2_6 |

TABLE E
List of coordinates for probes of pattern A from FIG. 8. The probes are
composed of combinations of complete or partial duplications of fragments of the GMC defined
in FIG. 2. The last column specifies which fragments of FIG. 2 compose each probe of pattern A.
Coordinates correspond to GRCh38/hg38 human reference database.

| Probe ID | Chromosome | Start | End | Probe color | Original Fragments |
|---|---|---|---|---|---|
| d13_dup | chr5 | 69535271 | 69555469 | Blue | d13_11, d13_12, d13_13 and |
| | | | | | d13_14 |
| d13_dup_2 | chr5 | 69555440 | 69574739 | Green | d13_7, d13_8, d13_9 and |
| | | | | | d13_10 |
| d13_dup_3 | chr5 | 69575107 | 69595239 | Red | d13_3, d13_4, d13_5 and |
| | | | | | d13_6 |
| d13_Naip_dup | chr5 | 69595660 | 69610510 | Blue | d13_1, d13_2a, d13_2b, |
| | | | | | d13_2c, d13_d, Naip_11, |
| | | | | | Naip_12 |
| Naip_dup | chr5 | 69610541 | 69628397 | Red | Naip_6, Naip_7, Naip_8, |
| | | | | | Naip_9, Naip_10 |
| d2_d7_dup | chr5 | 69666696 | 69685929 | Green | d7_1, d7_2, d2_5, d2_6 |
| d2_dup | chr5 | 69689523 | 69709640 | Cyan | d2_1, d2_2, d2_3, d2_4 |
| d6_dup | chr5 | 69861701 | 69886492 | Green | d6_3, d6_4, d6_5, d6_6 |
| d1_d6_dup | chr5 | 69729702 | 69749935 | Red | d6_1, d6_2, d1_5, d1_6 |
| d1_dup | chr5 | 69750054 | 69764937 | Blue | d1_2, d1_3, d1_4 |
| d12_dup | chr5 | 69764710 | 69785052 | Green | d12_1, d12_2, d12_3, d12_4, |
| | | | | | d1_1a, d1_1b |
| d1_dup_2 | chr5 | 69784339 | 69805209 | Blue | d1_1a, d1_1b, d1_2, d1_3, |
| | | | | | d1_4 |
| d1_d6_dup | chr5 | 69805328 | 69825541 | Red | d6_1, d6_2, d1_5, d1_6 |
| d6_dup | chr5 | 69825234 | 69845152 | Green | d6_3, d6_4, d6_5, d6_6 |
| d2_dup | chr5 | 69845219 | 69865706 | Cyan | d2_1, d2_2, d2_3, d2_4 |
| d2_d7_dup | chr5 | 69861701 | 69886492 | Green | d7_1, d7_2, d2_5, d2_6 |
| d7_dup | chr5 | 69885775 | 69905275 | Red | d7_3, d7_4, d7_5, d7_6 |
| d3_dup | chr5 | 69911365 | 69927200 | Blue | d3_1, d3_2, d3_3, d3_4 |
| d3_dup_2 | chr5 | 69927237 | 69937758 | Red | d3_5, d3_6 |
| d3_dup | chr5 | 69911365 | 69927200 | Blue | d3_1, d3_2, d3_3, d3_4 |
| d3_dup_2 | chr5 | 69927237 | 69937758 | Red | d3_5, d3_6 |
| d3_dup | chr5 | 69911365 | 69927200 | Blue | d3_1, d3_2, d3_3, d3_4 |

As shown herein, the invention provides a method that successfully mapped the region containing the SMN locus and which can also map other complex parts of a genome.

Spinal Muscular Atrophy (SMA) is an autosomal recessive motor neuron disease, which is the most common genetic cause of infant death, due to deletions/mutations in the SMN1 gene. Improvement of the detection, of SMA carrier is important in genetic counseling, especially in African-American population in which undetectable carriers are particularly frequent. The SMN1 gene, and its homologous SMN2 gene are localized on chromosome 5q13.2 in a complex region characterized by an inverted duplication of around 500 kb sequence. However, the precise mapping of this locus is extremely difficult with, the current technologies, such as sequencing or DNA microarray, due to high density of segmental duplications and other structural variations.

In order to precisely characterize the SMA locus, the inventors developed a specific GMC that cover the entire SMA region over 2 Mb. This GMC was hybridized on combed genomic DNA extracted from amnyocyte-derived cell cultures from African-American individuals. The image acquisition of fluorescent array signals was performed using an automated epifluorescence microscope, FiberVision®. After acquisition, SMA fluorescent array signals are pinpointed by the dedicated FiberStudio® software. The alignment of the different fluorescent array signals to the theoretical GMC deduced from the human genome reference sequence (GRCh38/hg38) reveals major discrepancies. First, it appeared that the two SAM genes were not in a head to-tail orientation as annotated but were in a head-to-head orientation. Moreover, a color pattern from the theoretical GMC was not observed in African-American samples indicating the absence of the corresponding sequence. The inventors also identified a repeat sequence with a variable number of repeated units located at the telomeric and/or centromeric regions indicating the presence of an unknown copy number variation sequence. Molecular Combing is a powerful technology that allowed the inventors to precisely and accurately map the SMA locus in the African-American population. This corrected map gives information that will be helpful in the development of relevant SMA screening tests for this population.

Moreover, the inventors developed a methodology to detect biomarkers for presence of duplication of SMN1 gene on the same allele, based on hybridizing probes from the SMA specific GMC, reconstituting each allele pattern from molecular combing data, and comparing allele patterns between a control group composed of individuals without the SMN1 duplication and a test group composed of individuals with the SMN1 duplication. They applied the methodology on African-American samples and discovered 17 potential patterns that are good biomarker candidates for cis-duplication of SMN1.

Terminology. Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not, constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise,

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by insertion of a space or underlined space before “www” and may be reactivated by removal of the space.

As used herein in the specification and claims, including as used in the examples and unless otherwise expressly specified, all numbers may, be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value for range of values), +/−1% of the stated value (or range of values), +/−2% of the stated value (or range of values), +/−5% of the stated value (or range of values), +/−10% of the stated value Or range of values), +/−15% of the stated value (or range of values). +/−20% of the stated value (or range of values), etc. Any numerical range recited herein is intended to include all sub-ranges subsumed therein.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms can and “may” and their variants are intended to be non-limiting, such that recitation that an embodiment can or may comprise certain elements or features does not exclude other embodiments of the present invention that do not contain those elements or features,

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention,

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

### BIBLIOGRAPHY

Lefebvre, S., L. Burglen, et al. Identification and characterization of a spinal muscular atrophy-determining gene. Cell 1995:80(1); 155-165.

Wirth B. Hahnen E, Morgan K, DiDonato C J, Dadze A, Rudnik-Schoneborn S. Simard L R, Zerres K, Burghes A H. Allelic association and deletions in autosomal recessive proximal spinal muscular atrophy: association of marker genotype with disease severity and candidate, cDNAs. Hum Mol Genet 1995; 4:1273-84

Melki J, Lefebvre S, Burglen L, Burlet P, Clermont O, MillasseauP, Reboullet S, Zeviani M, Le Paslier D, Cohen D, De novo and inherited deletions of the 5q13 region in spinal muscular atrophies, Science 1994; 264:1474-1477

Feldkötter M, Schwarzer V, Wirth. R, Wienker T F, Wirth. B. Quantitative analyses of SMN1 and SMN2 based on real-time lightCycler PCR: fast, and highly reliable carrier testing and prediction of severity of spinal muscular atrophy. Am J Hum Genet 2002; 70:358-368.

Huang C H, Chang Y Y, Chen C H, et al. Copy number analysis of survival motor neuron genes by multiplex ligation-dependent probe amplification. Genet Med 2007; 9:241-248

Anhuf D, Eggermann T, Rudnik-Schönebom S. Zerres K. Determination of SMN1 and SMN2 copy number using TaqMan technology, Hum Mutat 2003; 22:74-78

Hendrickson B C, Donohoe C, Akmaev V R, et al. Differences in SMN1 allele frequencies among ethnic groups within North America. J Med Genet 2009; 46:641-644

Luo, M., Liu, L., Peter, I., Zhu, J., Scott, S. A. Zhao, G., . . . & Edelmann, L. An Ashkenazi Jewish SMN1 haplotype specific to duplication alleles improves pan-ethnic carrier screening for spinal muscular atrophy. Genetics in Medicine, 2013:16(2), 149-156.

Sugarman, E. A., Nagan, N., Zhu, H., Akmaev, V. R., Zhou, Z., Rohlfs E. M., . . . & Allitto, B. A. Pan-ethnic carrier screening and prenatal diagnosis for spinal muscular atrophy: clinical laboratory analysis of >72400 specimens. European journal of human genetics, 2012:200), 27.

Bailey, J. A., Gu, Z., Clark, R. A., Reinert, K., Samonte, R. V., Schwartz, S., . . . & Eichler, E. E. Recent segmental duplications in the human genome. Science, 2002:297(5583), 1003-1007.

1. A method for detecting genomic DNA arrangement associated with a genetic disease, disorder or condition comprising
producing or providing a set of labelled probes covering a genomic region of interest that contains a gene of interest associated with the genetic disease, disorder or condition,
hybridizing the labelled probes to said region, wherein said probes are labelled with one of several different colors, wherein each color designates a different target or class of target sequences;
detecting a hybridization pattern formed on the genomic region of interest, and
reconstructing the hybridization patterns for each allele on the genomic region of interest;
comparing the hybridization, pattern of the labelled probes on the genomic region of interest between individuals in order to identify genetic direct or indirect biomarkers for the presence of carrier for the disease, disorder or condition.

2. The method of claim 1, wherein the genetic disease, disorder, or condition is spinal muscular atrophy (“SMA”) and the region of interest is an SMA locus.

3. The method of claim 2, wherein the labelled probes contain a color-coded probe that specifically recognizes SMN genes present in the control genomic DNA sequence which is a GRCh38/hg38 assembly or another control sequence spanning the SMA locus.

4. The method of claim 3, wherein the labelled probes further comprise bacterial artificial chromosome (BAC) or other orienting probes that when bound to the genomic region of interest orientate it with respect to a chromosomal centromere and telomere.

5. The method of claim 4, wherein the labelled probes thriller comprise probes that bind to repeat regions or other segments of the genomic region of interest.

6. The method of claim 5, wherein the genomic region of interest is obtained from a subject who has SMA, is a carrier of SMA, or who is otherwise at risk of having or carrying SMA.

7. The method of claim 6, wherein the genomic region of interest is obtained from germ cells, ovum, or sperm.

8. The method of claim 6, wherein the genomic region of interest is obtained in utero.

9. The method of claim 6, wherein the genomic region of interest is obtained from a prospective parent.

10. The method of claim 6, wherein the genomic region of interest is obtained from a subject having an African or African-American genetic profile.

11. The method of claim 6, further comprising diagnosing, counseling or treating a subject who has SMA, is a carrier of SMA, or who is otherwise at risk of having or carrying SMA.

12. A composition comprising a set of Genomic Morse Code (“GMC”) probes suitable for detecting and mapping SMN genes in a genomic DNA region of interest.

13. A kit comprising a set of labelled probes suitable for detecting and mapping SMN genes, a control genomic DNA sample or'providing a deduced or theoretical GMC pattern of a control DNA sample, instructions for use and packaging materials.

14. A method for characterizing at least one allele in a complex genetic region comprising:
selecting a genetic segment of interest,
producing or providing a set of labelled probes covering the genomic region of interest that contains an allele of interest,
hybridizing the labelled probes to said region, wherein said probes are labelled with one of several different colors, wherein each color designates a different target or class of target sequences;
detecting a hybridization pattern formed on the genomic region of interest, and
reconstructing the hybridization patterns for each allele on the genomic region of interest;
comparing the hybridization, pattern of the labelled probes on the genomic region of interest with a control hybridization pattern.

15. The method of claim 14, wherein the at least one allele is associated with a genetic disease, disorder or condition.

16. The method of claim 14, further comprising identifying at least one genetic biomarker for one or more alleles in the region of interest that distinguishes it from the corresponding region of interest in a group of control genomic profiles.

17. The method of claim 16, wherein the biomarker identifies a cis duplication of SMN 1.

18. A method for discovering an error in a sequence of a genomic region of interest described in a database comprising:
selecting a genetic segment of interest,
producing or providing a set of labelled probes covering the genomic region of interest that contains a segment to be inspected for errors,
hybridizing the labelled probes to said region, wherein said probes are labelled with one of several different colors, wherein each color designates a different target or class of target sequences;
detecting a hybridization pattern formed on the genomic region of interest, and
comparing the hybridization pattern of the labelled probes on the genomic region of interest with a theoretical hybridization pattern deduced from the database sequence to be inspected for errors;
identifying an error when a discrepancy is detected between the hybridization pattern of the genomic region of interest and the deduced hybridization pattern for the genomic region of interest from the database.

19. A method for identifying unpublished copy number variations (“CNV's”) in a sequence of a genomic region of interest comprising:
selecting a genetic segment of interest from genomic DNA to be tested for presence of copy number variations (“CNV's”);
producing or providing a set of labelled probes covering the genomic region of interest,
hybridizing the labelled probes to said region of interest, wherein said probes are labelled with one of several different colors, wherein each color designates a different target or class of target sequences;
detecting a hybridization pattern formed on the genomic region of interest, and
comparing the hybridization pattern of the labelled probes on the genomic region of interest with a theoretical hybridization pattern deduced from a control database sequence to be used as a referent for identifying unpublished CNVs; and
identifying a new CNV when a copy number of a particular segment of the region of interest differs from that a referent hybridization pattern.

20. The method of claim 19, wherein the referent hybridization pattern is deduced from a known genomic DNA sequence.

21. The method of claim 19, wherein the identified CNV is in the SMA genomic region.

22. The method of claim 17, where the biomarkers found are selected fragments of SMA region composed of combinations of complete or partial duplications of Genomic Morse Code (“GMC”) probes on SMA region.
